(19) United States
(12) Patent Application Publication
(10) Pub. No.: US 2025/0359748 A1
(43) Pub. Date: Nov. 27, 2025

(54) OTOSCOPE

(71) Applicant: GUANGZHOU LUXVISIONS INNOVATION TECHNOLOGY LIMITED, Guangzhou City (CN)

(72) Inventors: Chih-Ju Lin, Guangzhou City (CN); Chen-Fu Huang, Guangzhou City (CN)

(73) Assignee: GUANGZHOU LUXVISIONS INNOVATION TECHNOLOGY LIMITED, Guangzhou City (CN)

(21) Appl. No.: 19/008,523

(22) Filed: Jan. 2, 2025

(30) Foreign Application Priority Data
May 23, 2024 (CN) 202421141369.X

(51) Int. Cl. A61B 1/227; A61B 1/00; A61B 1/06

(52) U.S. Cl. CPC A61B 1/2275; A61B 1/00097; A61B 1/0676

(57) ABSTRACT

An otoscope is provided, which includes a sensing part. The sensing part includes a housing and a sensing head. The sensing head is disposed in the housing, and includes at least one light source and a sensing element. The light source is disposed on a front end face of the sensing head. The sensing element is disposed on the front end face. The light emitted by the light source can be fully used for illuminating the ear canal. Therefore, the energy consumption of the otoscope is low. The sensing element can completely receive the light reflected and scattered by the ear canal. Therefore, the sensing accuracy of the otoscope is high.

### CROSS-REFERENCE TO RELATED APPLICATION

This application claims the priority benefit of China application serial no. 202421141369.X, filed on May 23, 2024. The entirety of the above-mentioned patent application is hereby incorporated by reference herein and made a part of this specification.

### BACKGROUND

### Technical Field

The disclosure relates to an optical sensor, and particularly relates to an otoscope.

### Related Art

The traditional otoscope generally has a light source disposed inside a handle. The light source may be a light-emitting diode or an incandescent bulb, and transmit light to a sensing part through optical fibers. When the sensing part is inserted into the ear canal, light is projected onto the area to be sensed within the ear canal.

For some otoscopes, the housing of the sensing part is conical. The light-emitting diode is disposed inside the conical housing, and part of the light emitted therefrom may be blocked by the conical housing and unable to reach the area required to be sensed. A sensing element is also disposed inside the conical housing, which may cause part of the light reflected and/or scattered from the sensed area to be blocked by the housing and unable to reach the sensing element, and consequently reduce the sensing accuracy.

### SUMMARY

According to embodiments of the disclosure, an otoscope is provided, which does not require optical fibers for light transmission and has no concern of blocking illumination light and light to be sensed.

According to an embodiment of the disclosure, an otoscope is provided, which includes a sensing part. The sensing part includes a housing and a sensing head. The sensing head is disposed inside the housing, and includes at least one light source and a sensing element. The light source is disposed on a front end face of the sensing head. The sensing element is disposed on the front end face.

According to an embodiment of the disclosure, the otoscope further includes an air supply device. The air supply device includes an air supply tube and an air bag connected to the air supply tube. The air supply tube passes through a cavity of the sensing head, and an outlet of the air supply tube is located on the front end face.

According to an embodiment of the disclosure, the air supply tube includes stainless steel.

According to an embodiment of the disclosure, the otoscope further includes a lens disposed on the front end face of the housing. The lens is configured to converge light to be sensed onto the sensing element.

According to an embodiment of the disclosure, the otoscope further includes a main body part. The main body part includes a circuit board, and the sensing part is configured to be snap-fitted to the main body part to connect the at least one light source and the sensing element to the circuit board.

According to an embodiment of the disclosure, the sensing part is configured to be detached from the main body part.

According to an embodiment of the disclosure, the at least one light source is a light-emitting diode.

According to an embodiment of the disclosure, the at least one light source has a color temperature of 6500K or 3500K.

According to an embodiment of the disclosure, the housing of the sensing head is columnar.

According to an embodiment of the disclosure, the front end face has a diameter of 3.5 millimeters.

Based on the above, the otoscope provided by the embodiments of the disclosure configures the light sources on the front end face of the sensing head, thereby preventing the light from being blocked, and improving the illumination efficiency. The sensing element is also disposed on the front end face, which can prevent the light to be sensed from being blocked, and greatly improve the sensing accuracy.

To make the above features and advantages of the disclosure more understandable, exemplary embodiments will be described in detail below in conjunction with the accompanying drawings.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 shows a front view and a rear view of the otoscope according to an embodiment of the disclosure.

FIG. 2A is a schematic view showing the sensing part in FIG. 1, and FIG. 2B is a schematic view showing the sensing head of the sensing part in FIG. 2A.

FIG. 3A is a schematic view showing the otoscope according to another embodiment of the disclosure, FIG. 3B is a partial schematic view showing the otoscope in FIG. 3A, FIG. 3C is a partial schematic view showing the otoscope in FIG. 3A, and FIG. 3D is an enlarged view showing the front end face in FIG. 3B.

FIG. 4 is a schematic view showing the sensing part according to yet another embodiment of the disclosure.

### DETAILED DESCRIPTION OF DISCLOSED EMBODIMENTS

Exemplary embodiments of the disclosure will now be described in detail with reference to the accompanying drawings. The same reference numerals are used to denote the same or similar parts in the drawings and the description where possible.

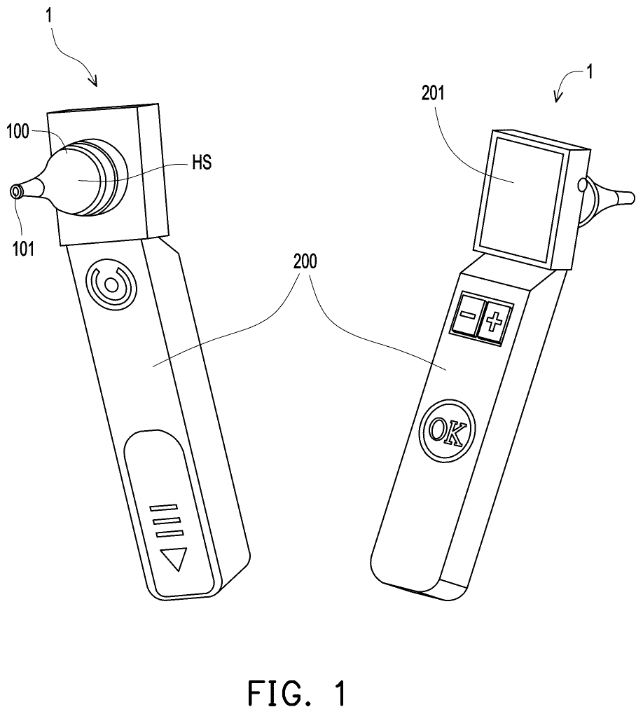

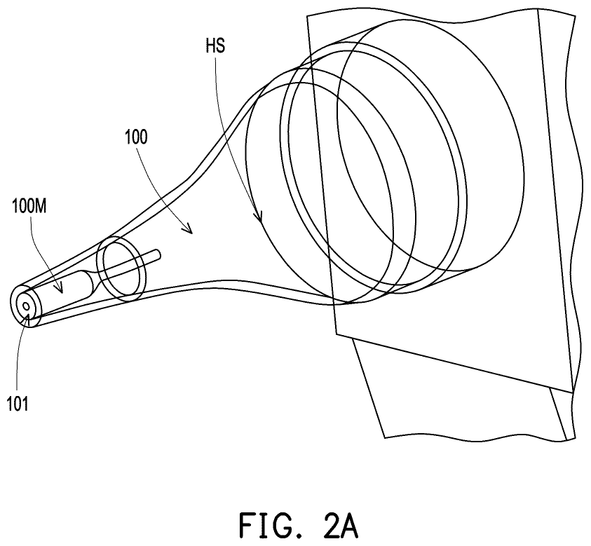

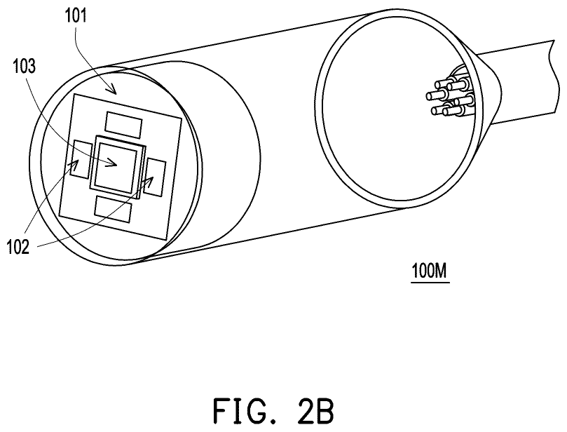

Referring to FIG. 1, FIG. 2A, and FIG. 2B, FIG. 1 shows a front view and a rear view of the otoscope according to an embodiment of the disclosure, FIG. 2A is a schematic view showing the sensing part in FIG. 1, and FIG. 2B is a schematic view showing the sensing head of the sensing part in FIG. 2A.

According to this embodiment, an otoscope 1 includes a sensing part 100 and a main body part 200. The sensing part 100 includes a housing HS and a sensing head 100M. The sensing head 100M is disposed inside the housing HS. The sensing head 100M has a front end face 101, and includes multiple light sources 102 and a sensing element 103 disposed on the front end face 101. There is a distance of approximately 500 micrometers between each light source 102 and the sensing element 103 to prevent a short circuit. The diameter of the front end face 101 may be, for example, 3.5 millimeters, but is not limited thereto. In some embodiments, the diameter of the front end face 101 may fall in a range of 4 millimeters to 5 millimeters.

The light sources 102 may be, for example, light-emitting diodes. The sensing element 103 may be, for example, a charge-coupled device (CCD) or a complementary metal-oxide-semiconductor (CMOS), and is disposed at the center of the front end face 101. The cavity of the sensing head 100M may be made of a biocompatible metal material, making it less likely to deform due to an external force. The outer housing thickness of the cavity of the sensing head 100M may roughly fall in a range of 300 micrometers to 800 micrometers.

The main body part 200 is provided with a circuit board and a processor therein, and the light sources 102 and the sensing element 103 may be connected to the circuit board and the processor inside the main body part 200 through power lines and signal lines that are accommodated in the cavity of the sensing head 100M.

In some embodiments, the sensing part 100 may be detached from the main body part 200, and may be attached to or detached from the main body part 200 by snap-fit. Specifically, the side of the sensing part 100 close to the main body part 200 may be provided with a circuit connector to connect the light sources 102 and the sensing element 103 to the circuit board and the processor.

It should be noted that, in the related art, one or more light sources 102 are disposed inside the housing HS, which may cause part of the light emitted by the light sources 102 to be blocked by the housing HS and unable to illuminate the area required to be sensed. In contrast, in the embodiment of the disclosure, the light sources 102 are disposed on the front end face 101 of the sensing head 100M, which can prevent the light from being blocked and thereby improve the illumination efficiency. Further, referring to FIG. 2B, multiple light sources 102 are disposed on the front end face 101 in this embodiment, and the light sources 102 are symmetrically arranged on the front end face 101, thereby providing uniform illumination in all directions.

In addition, in the related art, the sensing element 103 is also disposed inside the housing HS, which may cause part of the light reflected and/or scattered from the sensed area to be blocked and unable to reach the sensing element 103, and consequently reduce the sensing accuracy. In contrast, in the embodiment of the disclosure, the sensing element 103 is disposed on the front end face 101 of the sensing head 100M, which can prevent the light to be sensed from being blocked, and thereby greatly improve the sensing accuracy and reduce energy loss to achieve energy-saving effects.

In some embodiments, the light sources 102 and the sensing element 103 may be encapsulated by an encapsulation material. In some embodiments, a lens may also be disposed at the front end of the encapsulation to converge the light to be sensed that is reflected and/or scattered from the sensed area onto the sensing element 103, thereby improving the sensing accuracy of the optical otoscope 1.

In some embodiments, the light sources 102 have a color temperature of 6500K, with an emission wavelength range of approximately 400 nm to 750 nm, and have local peaks at 460 nm and 575 nm. However, the disclosure is not limited thereto. In some embodiments, the light sources 102 may have a color temperature of 3500K.

To fully explain various forms of implementation of the disclosure, other embodiments of the disclosure will be described below. It should be noted here that the following embodiments adopt the reference numerals and partial content of the preceding embodiments, where the same reference numerals are used to denote the same or similar elements, and description of the same technical content will be omitted. Please refer to the preceding embodiments for the omitted content, which will not be repeated here.

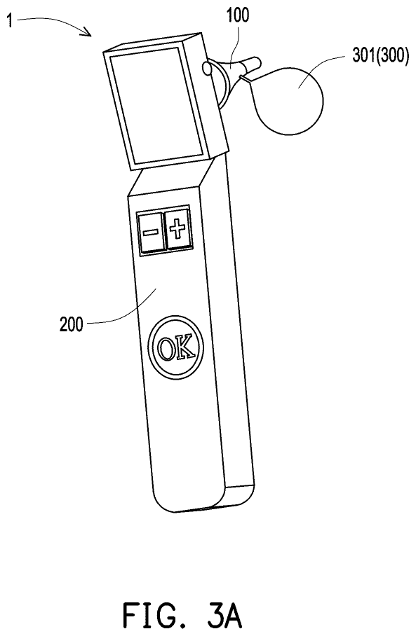

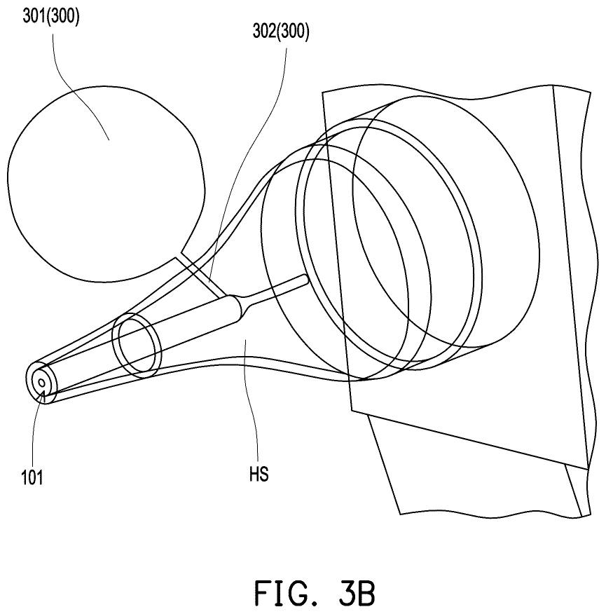

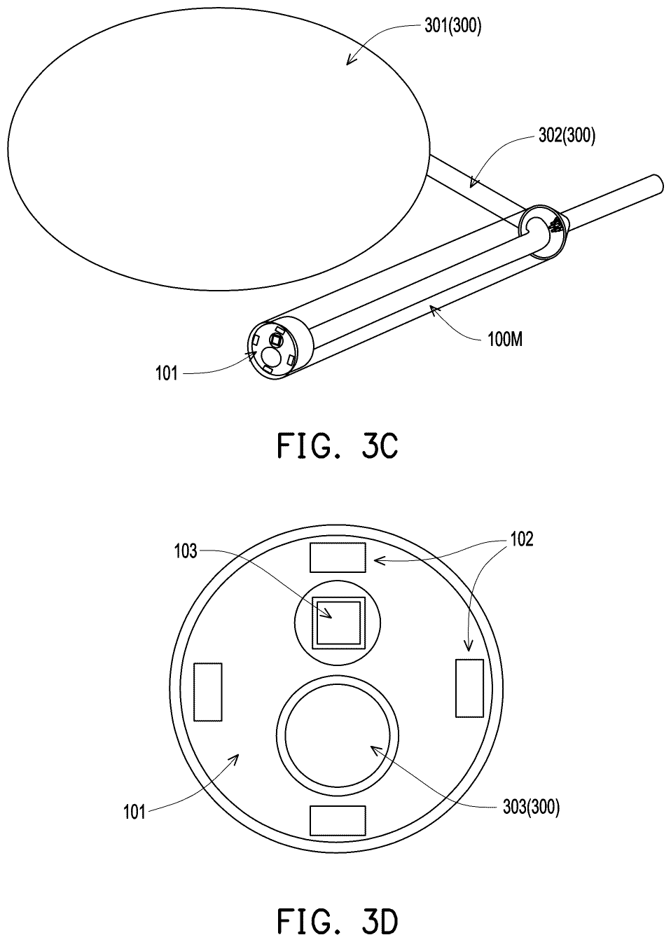

Referring to FIG. 3A to FIG. 3D, FIG. 3A is a schematic view showing the otoscope according to another embodiment of the disclosure, FIG. 3B is a partial schematic view showing the otoscope in FIG. 3A, FIG. 3C is a partial schematic view showing the otoscope in FIG. 3A, and FIG. 3D is an enlarged view showing the front end face in FIG. 3B.

According to this embodiment, an otoscope 1 includes a sensing part 100, a main body part 200, and an air supply device 300. The components and structures of the sensing part 100 and the main body part 200 are substantially the same as those in the preceding embodiments, and will not be repeated here.

The air supply device 300 of this embodiment is configured to provide gas into the ear cavity of the user to test the balance response of the user. Specifically, the air supply device 300 includes an air supply tube 302 and an air bag 301 connected to the air supply tube 302. The air supply tube 302 also passes through the cavity of the sensing head 100M, and an air supply tube outlet 303 of the air supply tube 302 is located on the front end face 101. In some embodiments, the air supply tube 302 may include stainless steel. When the front end face 101 of the sensing head 100M is placed into the ear cavity of the user, gas can be pushed into the air supply tube 302 by pressing the air bag 301, so as to eject the gas from the air supply tube outlet 303.

It should be noted that, in the related art, the light sources 102 and the sensing element 103 are disposed inside the housing HS. If the air supply tube 302 is also disposed inside the housing HS, the air supply tube 302 may block the light emitted by the light sources 102, or block the light to be sensed that travels toward the sensing element 103. In contrast, according to the disclosure, the light sources 102 and the sensing element 103 can be disposed on the front end face 101, which allows the air supply tube 302 to be disposed inside the housing HS without blocking the light.

Referring to FIG. 3D, since the air supply tube outlet 303 is disposed on the front end face 101, the sensing element 103 of this embodiment is not disposed at the center of the front end face 101. In this embodiment, as shown in FIG. 3D, four light sources 102 are disposed on the front end face 101, and the four light sources 102 are symmetrically arranged on the front end face 101. Nevertheless, the disclosure is not limited thereto. In an embodiment, only two light sources 102 may be disposed on the front end face 101, and the two light sources 102 are symmetrically arranged on the front end face 101 with respect to the center of the front end face 101. In an embodiment, only one light source 102 may be disposed on the front end face 101.

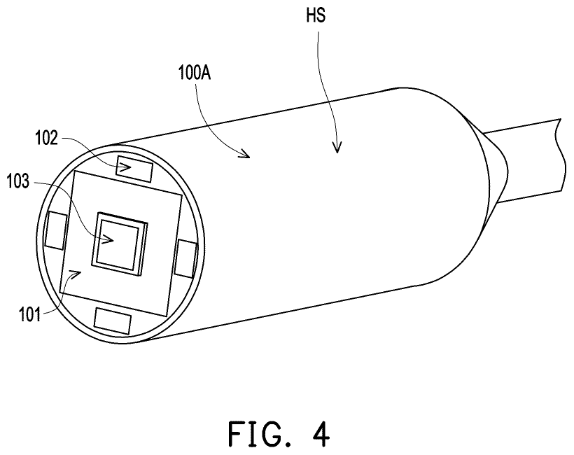

Referring to FIG. 4, FIG. 4 is a schematic view showing the sensing part according to an embodiment of the disclosure. In this embodiment, the housing HS of a sensing part 100A is columnar, which is different from the conical housing HS shown in the preceding embodiments (as shown in FIG. 1 and FIG. 3B). Specifically, since the disclosure configures the light sources 102 and the sensing element 103 on the front end face 101 compared to the related art, the interior of the housing HS of the sensing part 100A does not need space for accommodating the light sources 102 and the sensing element 103. Therefore, the housing HS of the sensing part 100A is not required to have a conical shape as shown in the preceding embodiments.

In summary, the otoscope provided by the embodiments of the disclosure configures the light sources on the front end face of the sensing head, thereby preventing the light from being blocked, and improving the illumination efficiency. The sensing element is also disposed on the front end face, which can prevent the light to be sensed from being blocked, and greatly improve the sensing accuracy.

It will be apparent to those skilled in the art that various modifications and changes can be made to the disclosed embodiments without departing from the scope or spirit of the disclosure. In view of the foregoing, it is intended that the disclosure covers modifications and changes that fall within the scope of the appended claims and their equivalents.

What is claimed is:

1. An otoscope, comprising:
a sensing part which comprises:
a housing; and
a sensing head disposed in the housing and comprising:
at least one light source disposed on a front end face of the sensing head; and
a sensing element disposed on the front end face.

2. The otoscope according to claim 1, further comprising an air supply device,
wherein the air supply device comprises an air supply tube and an air bag connected to the air supply tube, wherein the air supply tube passes through a cavity of the sensing head, and an outlet of the air supply tube is located on the front end face.

3. The otoscope according to claim 2, wherein the air supply tube comprises stainless steel.

4. The otoscope according to claim 1, further comprising a lens disposed on the front end face of the hosing,
wherein the lens is configured to converge light to be sensed onto the sensing element.

5. The otoscope according to claim 1, further comprising a main body part,
wherein the main body part comprises a circuit board, and the sensing part is configured to be snap-fitted to the main body part to connect the at least one light source and the sensing element to the circuit board.

6. The otoscope according to claim 5, wherein the sensing part is configured to be detached from the main body part.

7. The otoscope according to claim 1, wherein the at least one light source is a light-emitting diode.

8. The otoscope according to claim 7, wherein the at least one light source has a color temperature of 6500K or 3500K.

9. The otoscope according to claim 1, wherein the housing of the sensing head is columnar.

10. The otoscope according to claim 1, wherein the front end face has a diameter of 3.5 millimeters.
